(19) United States
(12) Patent Application Publication
(10) Pub. No.: US 2024/0325381 A1
(43) Pub. Date: Oct. 3, 2024

(54) USE OF FLUNARIZINE AND METHOD FOR CONTROLLING NUMBER OF INTERCELLULAR MITOCHONDRIA

(71) Applicant: GUANGZHOU INSTITUTES OF BIOMEDICINE AND HEALTH, CHINESE ACADEMY OF SCIENCES, Guangzhou (CN)

(72) Inventors: Xingguo LIU, Guangzhou (CN); Feixiang BAO, Guangzhou (CN)

(21) Appl. No.: 18/740,547

(22) Filed: Jun. 12, 2024

Related U.S. Application Data
(63) Continuation of application No. PCT/CN2022/110893, filed on Aug. 8, 2022.

(30) Foreign Application Priority Data
Dec. 27, 2021 (CN) 202111612903.1

(51) Int. Cl. A61K 31/495; A61P 43/00

(52) U.S. Cl. CPC A61K 31/495; A61P 43/00

(57) ABSTRACT

The present application relates to uses of flunarizine and a method for controlling the number of intercellular mitochondria. The uses include use of the flunarizine in removing intracellular mitochondria, and use of the flunarizine in the preparation of a medicament for preventing and/or treating a disease associated with mitochondrial abnormality. The method includes: controlling the total amount of mitochondria in the brain by taking the flunarizine; contacting the flunarizine with cells to be treated, and controlling the total amount of the intercellular mitochondria based on the influence of the contact time on the removal amount of the mitochondria.

### CROSS-REFERENCE TO RELATED APPLICATIONS

This application is a continuation of International Application No. PCT/CN2022/110893, filed on Aug. 8, 2022, which claims priority to Chinese Patent Application No. 202111612903.1, filed on Dec. 27, 2021. The disclosures of the aforementioned applications are hereby incorporated by reference in their entireties.

### FIELD

The present disclosure relates to the field of biotechnology, particularly, to uses of flunarizine and a method for controlling the number of intracellular mitochondria, and more particularly, to uses of flunarizine, a single-dose flunarizine, a method for removing intracellular mitochondria, a method for promoting mitochondrial efflux, a method for controlling a removal number of mitochondria, use of flunarizine in the preparation of a kit, and cells.

### BACKGROUND

Mitochondria are organelles surrounded by two membranes which exist in most cells, and they are of structures producing energy in cells and the main place for cells to carry out aerobic respiration. Mitochondria are also organelles closely related to energy metabolism. Both cell survival (oxidative phosphorylation) and cell death (apoptosis) are related to mitochondrial function, and especially, abnormal oxidative phosphorylation of the respiratory chain is related to many human diseases.

Therefore, it is urgent to find a method for regulating the intracellular mitochondria.

### SUMMARY

The present disclosure provides a method for removing intracellular mitochondria. The method includes contacting flunarizine with cells to be treated.

The present disclosure further provides a method for preventing and/or treating a disease associated with mitochondrial abnormality. The method includes administrating flunarizine to a subject in need thereof.

The present disclosure further provides a single-dose flunarizine formulation. According to an embodiment of the present disclosure, 10 to 30 μM, and preferably, 15 to 25 μM of flunarizine is used.

The present disclosure further provides a method for promoting mitochondrial efflux. The method includes: contacting flunarizine with the cells to be treated.

The present disclosure further provides a method for controlling a removal amount of mitochondria. The method includes: contacting flunarizine with the cells to be treated; and controlling the removal amount of intracellular mitochondria based on the contact time.

The present disclosure further provides a cell with the number of intracellular mitochondria lower than the number of intracellular mitochondria in a normal cell. Exogenous parkin is not expressed in the cell, and the cell is obtained by the method described in the fifth aspect.

### BRIEF DESCRIPTION OF THE DRAWINGS

The above and/or additional aspects and advantages of the present disclosure will become apparent and comprehensible based on the description of the embodiments in conjunction with the following drawings.

FIG. 1 is a schematic diagram of removing intracellular mitochondria by flunarizine according to an embodiment of the present disclosure;

FIG. 2 illustrates contents of intracellular mitochondria at different time points in Example 1 of the present disclosure;

FIG. 3 illustrates results of the clearance of mitochondria by flunarizine, independent of mitophagy, in Example 2 of the present disclosure;

FIG. 4 illustrates results of the clearance of mitochondria by flunarizine, accomplished by mitochondrial efflux, in Example 3 of the present disclosure;

FIG. 5 illustrates results of the clearance of mitochondria in brains of mice by flunarizine in Example 4 of the present disclosure;

FIG. 6 illustrates results of influences of flunarizine on different neurons and glial cells in the brains of the mice in Example 5 of the present disclosure; and

FIG. 7 illustrates results of contents of different types of proteins on mitochondria cultured with or without flunarizine in Example 6 of the present disclosure.

### DETAILED DESCRIPTION

The present disclosure aims to solve at least one of the technical problems existing in the prior art at least to some extent. To this end, the present disclosure provides use of flunarizine and a method for controlling the number of intracellular mitochondria. According to the present disclosure, flunarizine can remove the intracellular mitochondria.

The present disclosure is based on the Applicant's following findings.

Mitochondria are organelles closely related to energy metabolism. Both cell survival (oxidative phosphorylation) and cell death (apoptosis) are related to mitochondrial function, and especially, abnormal oxidative phosphorylation of the respiratory chain is related to many human diseases, for example, mitochondrial myopathy, mitochondrial encephalomyopathy, and neurodegenerative disease. Among them, the etiology of the neurodegenerative disease (such as Alzheimer's disease) was previously considered to be the accumulation of damaged mitochondria and the build-up of proteins, which eventually lead to neuron degeneration. Therefore, the removal of damaged mitochondria is of great help to improve the symptoms of neurodegenerative diseases, and at the same time, it also has become a potential drug target for the treatment of neurodegenerative diseases. The etiology of mitochondrial myopathy and mitochondrial encephalomyopathy was previously considered to be the deletion or point mutation of mitochondrial DNA, which causes the malfunction of enzymes or carriers necessary for encoding the mitochondrial oxidative metabolism process. Thus, glycogen and fatty acids are inhibited to enter the mitochondria to fully utilize and produce enough ATP, resulting in a disorder of energy metabolism and the development of complex clinical symptoms. Therefore, the removal of mitochondria and the introduction of exogenous mitochondria containing normal DNA can repair the mutation of mitochondrial DNA and can be conducive to the improvement or treatment of mitochondrial myopathy and mitochondrial encephalomyopathy.

At present, the existing technical means is to remove intracellular mitochondria based on the exogenous expression of parkin and the uncoupling agent FCCP. Specifically, through overexpression of the ubiquitin E3 ligase parkin protein, in combination with short-term treatment of mitochondrial uncoupling agent FCCP, the autophagosome is stimulated to mediate lysosome to eliminate mitochondria in cells, so as to achieve mitophagy. However, in such a method, overexpression through virus insertion into the genome may affect the stability of the nucleus.

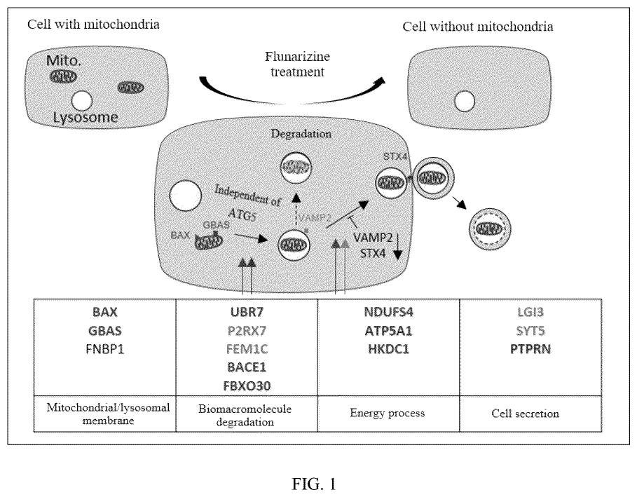

In order to solve the above problems, the Applicant investigated various physiological and pathological functions of mitochondria by controlling the content of intracellular mitochondria through in vivo and in vitro experiments. The Applicant found that, after the flunarizine (chemical formula C₂₆H₂₆F₂N₂) was in contact with the cells to be treated, the intracellular mitochondria could enter the lysosome independently of mitophagy, and then they could be discharged out of the cells through the lysosomes, thereby completely or partially removing the mitochondria in the cells. The schematic diagram of mitochondrial removal is illustrated in FIG. 1. Moreover, the removal amount of intracellular mitochondria can be controlled by controlling the contact time between flunarizine and the cells to be treated. When the contact time is more than 3 days, the intracellular mitochondria can be completely removed, to prepare cells without mitochondria. Particularly, for a patient with mitochondrial DNA mutations, the mitochondria can be removed by this method, and exogenous mitochondria containing normal DNA can be introduced to repair mitochondria. According to the method of the present disclosure, the cells are not required to express exogenous genes, having insignificant influence on the nucleus, and they do not rely on mitophagy, which can broaden the way for the research and analysis of mitochondria.

In the first aspect of the present disclosure, the present disclosure proposes use of flunarizine in removing intracellular mitochondria. The Applicant found through a huge amount of experiments that, after the flunarizine was in contact with the cells to be treated, the mitochondria can enter the lysosomes, and then they can be discharged out of the cells through the lysosomes, thereby completely or partially removing the mitochondria in the cells. In the present disclosure, by using flunarizine, the cells are not required to express exogenous genes, having insignificant influence on the nucleus, and they do not rely on mitophagy, which can broaden the way for the research and analysis of mitochondria.

According to embodiments of the present disclosure, the above use can further include at least one of the following additional technical features.

According to an embodiment of the present disclosure, the mitochondria include mitochondria with a DNA mutation and/or mitochondria without a DNA mutation. The above mitochondria in the cells can be effectively removed by contacting the flunarizine with the cells.

In the second aspect of the present disclosure, the present disclosure proposes use of flunarizine in the preparation of a medicament for preventing and/or treating a disease associated with mitochondrial abnormality. The Applicant has found through experiments that the use of a medicament containing flunarizine can remove the intracellular mitochondria, thereby effectively preventing and treating the above diseases related to mitochondrial gene mutation or mitochondrial abnormality.

According to the embodiments of the present disclosure, the above use may further include at least one of the following additional technical features.

According to an embodiment of the present disclosure, the mitochondrial abnormality includes mitochondrial gene mutation and/or mitochondrial dysfunction.

According to an embodiment of the present disclosure, the diseases related to mitochondrial gene mutation include: mitochondrial myopathy and mitochondrial encephalomyopathy. The Applicant found through experiments that the above diseases can be effectively treated by using the medicament containing flunarizine.

According to an embodiment of the present disclosure, the diseases related to mitochondrial dysfunction include: neurodegenerative disease. The Applicant found through experiments that the above disease can be effectively treated by using the medicament containing flunarizine.

According to an embodiment of the present disclosure, the neurodegenerative disease includes at least one selected from Alzheimer's disease, Parkinson's disease, Huntington's disease, amyotrophic lateral sclerosis, spinocerebellar ataxia, lobar sclerosis, cerebral ischemia, brain injury, and epilepsy. The Applicant found through experiments that the above diseases can be effectively treated by using the medicament containing flunarizine.

According to an embodiment of the present disclosure, the mitochondrial encephalomyopathy includes at least one selected from myoclonic epilepsy with red fiber disease (MERRF syndrome), mitochondrial encephalomyopathy with lactic acidosis and stroke-like episodes syndrome (MELAS), Leber's hereditary optic neuropathy (LHON), ophthalmoplegia syndrome (KSS syndrome), subacute necrotizing encephalopathy (Leigh syndrome), familial primary progressive cerebral gray matter atrophy (Alpers disease), kinky-hair syndrome (Menke's disease), and retinitis pigmentosa ataxia peripheral neuropathy (NARP). The Applicant found through experiments that the above diseases can be effectively treated by using the medicament containing flunarizine.

According to an embodiment of the present disclosure, a working concentration of flunarizine ranges from 10 to 30 μM, and preferably, 15 to 25 μM. The above optimal working concentrations were obtained by the Applicant through a large number of experiments, for improving the therapeutic effect on diseases related to mitochondrial gene mutation or mitochondrial dysfunction.

It should be noted that the “working concentration” refers to the final concentration of flunarizine in the culture environment of the cells to be treated.

In a third aspect of the present disclosure, the present disclosure proposes a single-dose flunarizine formulation. According to an embodiment of the present disclosure, 10 to 30 μM, and preferably, 15 to 25 μM of flunarizine is used. It should be noted that the “single dose” refers to the dose used by an adult at one time. The Applicant found that the use of the above single-dose formulation can effectively treat the above diseases related to mitochondrial gene mutation or mitochondrial dysfunction.

In a fourth aspect of the present disclosure, the present disclosure proposes a method for removing intracellular mitochondria. According to an embodiment of the present disclosure, the method includes: contacting flunarizine with the cells to be treated. The Applicant have found through experiments that parts or all of the mitochondria in the cells can be removed by using the above method. Moreover, according to the method of the present disclosure, the cells are not required to express exogenous genes, having insignificant influence on the nucleus, and they do not rely on mitophagy, which can broaden the way for the research and analysis of mitochondria.

According to the embodiments of the present disclosure, the above method may further include at least one of the following additional technical features.

According to an embodiment of the present disclosure, the concentration of flunarizine in the contact environment ranges from 10 to 30 μM, and preferably, 15 to 25 μM. The above optimal concentrations were obtained by the Applicant through a large number of experiments, for improving the removal effect of the intracellular mitochondria. Moreover, the Applicant have found through experiments that, when the concentration of flunarizine is excessively high, the cell death rate will be increased; and when the concentration of flunarizine is excessively low, the removal effect on mitochondria is poor.

According to an embodiment of the present disclosure, the cells to be treated are plated in a cell culture plate in advance.

According to an embodiment of the present disclosure, the surface coverage of the cells to be treated in the cell culture plate ranges from 75% to 85%. The above optimal concentrations were obtained by the Applicant through a large number of experiments, for improving the removal effect of the intracellular mitochondria. The Applicant have found through experiments that the cell density has a great influence on the reaction. When the cell density is excessively high, the removal effect of mitochondria is poor. When the cell density is excessively low, the cell death rate is excessively high, which will affect the experimental results.

According to an embodiment of the present disclosure, the contact time ranges from 0.1 to 5 days, and preferably, 0.5 to 3 days. The above optimal contact time were obtained by the Applicant through a large number of experiments, for improving the removal effect of the intracellular mitochondria.

In the fifth aspect of the present disclosure, the present disclosure provides a method for promoting mitochondrial efflux. According to an embodiment of the present disclosure, the method includes: contacting flunarizine with the cells to be treated. The Applicant have found through experiments that, such a method is independent of mitophagy, and by contacting flunarizine with the cells to be treated, mitochondria can enter lysosomes to enable the mitochondria to be discharged out of the cells through the lysosomes, thereby completely or partially removing the mitochondria in the cells. In this way, the way for the research and analysis of mitochondria can be broaden.

According to the embodiments of the present disclosure, the above method may further include at least one of the following additional technical features.

According to an embodiment of the present disclosure, the concentration of flunarizine in the contact environment ranges from 10 to 30 μM, and preferably, 15 to 25 μM. The above optimal concentrations were obtained by the Applicant through a large number of experiments, for enhancing the efficiency of discharging the intracellular mitochondria out of the cell.

According to an embodiment of the present disclosure, the cells to be treated are plated in a cell culture plate in advance.

According to an embodiment of the present disclosure, the surface coverage of the cells to be treated in the cell culture plate ranges from 75% to 85%.

According to an embodiment of the present disclosure, the contact time ranges from 0.1 to 5 days, and preferably, 0.5 to 3 days, which is conducive to the discharge of the intracellular mitochondria out of the cells.

In a sixth aspect of the present disclosure, the present disclosure provides a method for controlling a removal amount of mitochondria. According to an embodiment of the present disclosure, the method includes: contacting flunarizine with the cells to be treated; and controlling the removal amount of intracellular mitochondria based on the contact time. The Applicant have found through experiments that such a method can control the removal amount of intracellular mitochondria by contacting flunarizine with the cells to be treated, and then discharging the mitochondria out of the cells through lysosomes, and controlling the contact time.

According to the embodiments of the present disclosure, the above method can further include at least one of the following additional technical features.

According to an embodiment of the present disclosure, the removal amount of intracellular mitochondria is not higher than ⅓ for the contact time shorter than 1 day. Thus, ⅓ of the intracellular mitochondria can be removed.

According to an embodiment of the present disclosure, the removal amount of intracellular mitochondria ranges from ⅓ to ½ for the reaction time of 1 to 2 days. Thus, ⅓ to ½ of the intracellular mitochondria can be removed.

According to an embodiment of the present disclosure, the removal amount of intracellular mitochondria is higher than ½ for the reaction time more than 2 days. Thus, more than ½ of the intracellular mitochondria can be removed.

According to an embodiment of the present disclosure, the intracellular mitochondria are completely removed for the reaction time more than 3 days. Thus, the intracellular mitochondria can be completely removed.

According to an embodiment of the present disclosure, the concentration of flunarizine in the contact environment ranges from 10 to 30 μM, and preferably, 15 to 25 μM. The above optimal concentrations are obtained by the Applicant through a large number of experiments, for improving the removal effect of the intracellular mitochondria.

According to an embodiment of the present disclosure, the cells to be treated are plated in a cell culture plate in advance.

According to an embodiment of the present disclosure, the surface coverage of the cells to be treated in the cell culture plate is 75% to 85%. Thus, the removal effect of the intracellular mitochondria is better.

According to an embodiment of the present disclosure, the contact time is 0.1 to 5 days, and preferably, 0.5 to 3 days, which can improve the removal effect of the intracellular mitochondria.

In a seventh aspect of the present disclosure, the present disclosure proposes use of flunarizine in the preparation of a kit for removing intracellular mitochondria. The intracellular mitochondria in cells can be removed by using the above kit.

In an eighth aspect of the present disclosure, the present disclosure proposes a cell with the number of intracellular mitochondria lower than the number of intracellular mitochondria in a normal cell. According to an embodiment of the present disclosure, exogenous parkin is not expressed in the cell, and the cell is obtained by the method described in the fifth aspect. Thus, by using the aforementioned method, the mitochondria can be discharged out of the cell through the lysosome and thus be removed, and accordingly, the cell with the lower number of mitochondria than the normal number of mitochondria can be obtained. That is, the number of mitochondria in the obtained cell is lower than that in the cell of the same kind without pathological changes or treatment, and exogenous parkin is not expressed in the cell.

It should be noted that the normal number of mitochondria refers to the number of mitochondria in a cell in healthy physiological state. The number of mitochondria in different cells varies. For example, the normal number of mitochondria in a liver cell ranges from 1,000 to 2,000.

It should be noted that the terms “first” and “second” are only used for descriptive purposes, and cannot be understood as indicating or implying relative importance or implicitly indicating the number of indicated technical features. Thus, a feature defined as “first” or “second” may explicitly or implicitly include one or more of such features. Further, in the description of the present disclosure, unless otherwise specified, “plurality” means two or more.

The solutions of the present disclosure will be explained below in conjunction with examples. Those skilled in the art will understand that the following examples are only for illustrating the present disclosure and should not be considered as limiting the scope of the present disclosure. The specific techniques or conditions not specified in the examples are those described in literature in the field or product instructions will be followed. The manufacturer of the reagents or instruments used, when not specified, are all conventional products that can be purchased commercially.

It should be noted that the neural precursor cells and mouse embryonic fibroblasts (MEF cells) in the following examples are both cultured in commercially available serum-free N2B27 medium. Mito-X labeling refers to that the intracellular mitochondria have an X group (which can be any group) labeling thereon. LC3-X labeling refers to that the autophagy marker LC3 protein in the cell has an X group (which can be any group) labeling thereon. LAMP1-X labeling refers to that the lysosome-associated membrane protein 1 (LAMP1) in the cell has an X group (which can be any group) labeling. FNZ-indicates the control group without flunarizine treatment, and FNZ+ indicates the experimental group with flunarizine treatment.

### Example 1: Research on Clearance of Mitochondria by Flunarizine

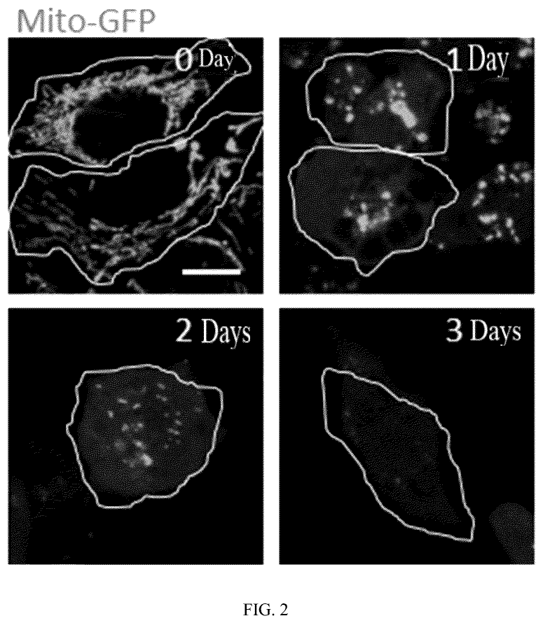

Mito-GFP-labeled neural precursor cells were plated on a 6-well plate, and the surface coverage of the neural precursor cells on the 6-well plate was 80%. After overnight culture, the neural precursor cells were treated with flunarizine for 3 days, and the final concentration of flunarizine was 15 μM. Then, the cells were detected with a confocal microscopy and the clearance efficiency of the mitochondrial was observed. The specific results are illustrated in FIG. 2. The results indicate that the removal amount of the intracellular mitochondria was different at different treatment times, and the intracellular mitochondria were substantially completely removed after 3 days of treatment.

### Example 2: Research on Clearance of Mitochondria by Flunarizine Independent of Mitophagy

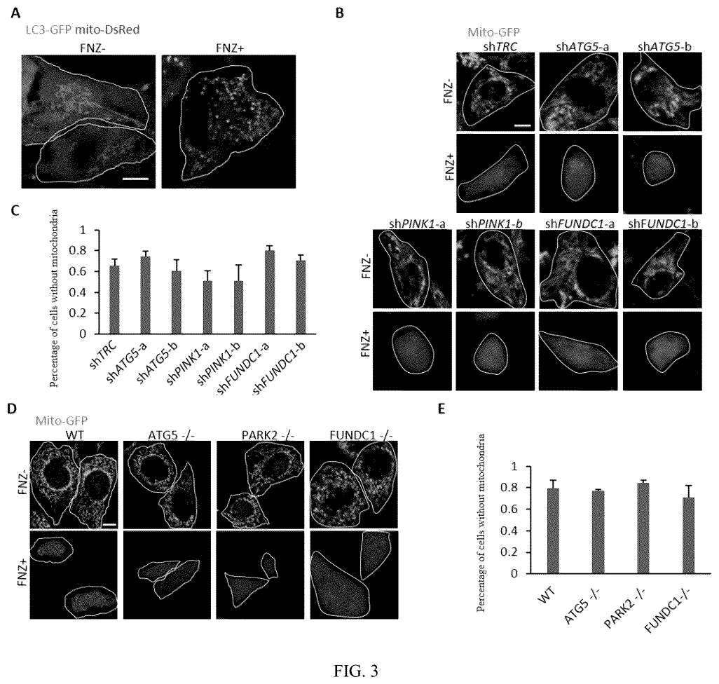

The neural precursor cells labeled with LC3-GFP and mito-DsRed were plated on the glass slides, and the surface coverage of the neural precursor cells on the glass slides was 80%. After overnight culture, the neural precursor cells were treated with flunarizine for 12 hours, and the final concentration of flunarizine was 15 μM. Then, the results were detected with a confocal microscope. The specific results are illustrated in FIG. 3. The results indicate that the mitochondria treated with flunarizine were not co-localized with LC3-GFP labeled autophagosomes, substantiating that the process of mitochondrial clearance was independent of mitophagy.

The corresponding classic mitophagy core genes (including shTRC, shATG5-a, shATG5-b, shPINK1-a, shPINK1-b, shFUNDC1-a, and shFUNDC1-b) in mito-GFP-labeled neural precursor cells were knocked down. Then, the cells were plated on a glass slide, the surface coverage of neural precursor cells on the glass slide was 80%. After overnight culture, the neural precursor cells were treated with flunarizine for 3 days, and the final concentration of flunarizine was 15 μM. Thereafter, the cells were detected with a confocal microscope. The specific results are illustrated in FIG. 3B and FIG. 3C. The results indicate that knockdown of related genes could not inhibit mitochondrial clearance.

Mito-GFP-labeled mouse embryonic fibroblasts (MEF cells) were plated on a glass slide. The corresponding classic mitophagy core genes (including WT, ATG5, PARK2, and FUNDC1) in the MEF cells were knocked off. The surface coverage of the cells on the glass slide was 80%. After overnight culture, the MEF cells were treated with flunarizine for 3 days, and the final concentration of flunarizine was 15 μM. Then, the cells were detected with a confocal microscope. The specific results are illustrated in FIG. 3D and FIG. 3E. The results indicate that knockdown of mitophagy-related genes cannot inhibit mitochondrial clearance.

### Example 3: Research on Clearance of Mitochondria by Flunarizine with Process of Mitochondria Entering Lysosomes

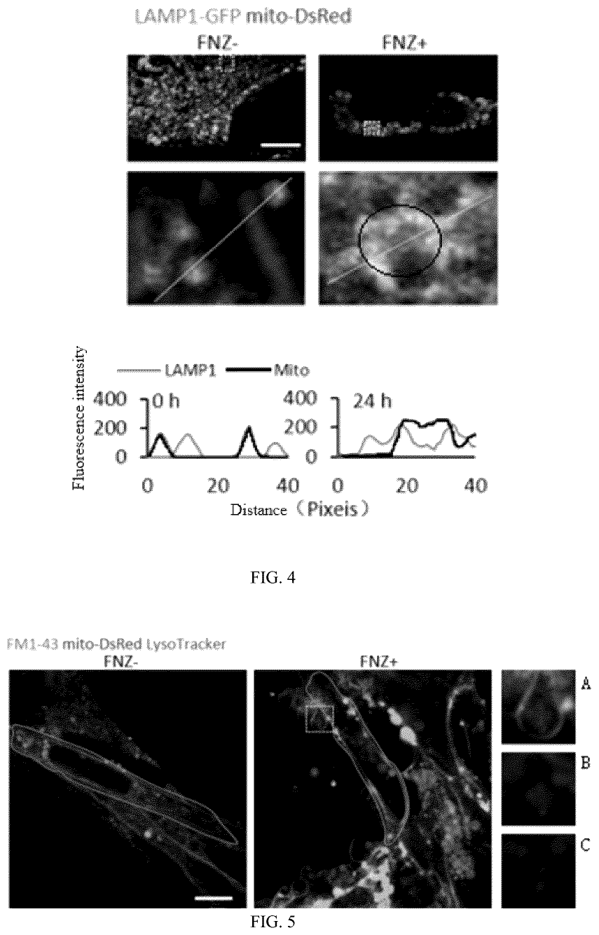

The neural precursor cells labeled with LAMP1-GFP and mito-DsRed were plated on a glass slide, and the surface coverage of the neural precursor cells on the glass slide was 80%. After overnight culture, the neural precursor cells were treated with flunarizine for 12 hours, and the final concentration of flunarizine was 15 μM. Then, the results were detected with a confocal microscope. The specific results are illustrated in FIG. 4. In FIG. 4, LAMP1 and mito do not overlap in the FNZ-figures, and LAMP1 and mito overlap (the inside of the circular frame is the overlapping part) in the FNZ+ figures. The results indicate that the flunarizine-treated mitochondria were wrapped by lysosomes.

### Example 4: Research on Clearance of Mitochondria by Flunarizine with Process of Mitochondrial Efflux

Mito-DsRed-labeled neural precursor cells were plated on a glass slide, and the surface coverage of the neural precursor cells on the glass slide was 80%. After overnight culture, the neural precursor cells were treated with flunarizine for 24 hours. The final concentration was 15 μM. The lysosomes were labeled with Lysotraker DeepRed, the cell membranes were labeled with FM1-43, and then the results were detected with a confocal microscope. The specific results are illustrated in FIG. 5. In FIG. 5, no overlapping part of mitochondria, lysosomes and cell membranes existed in the FNZ-figure; and the boxed part in the FNZ+ figure was the overlapping part of mitochondria, lysosomes and cell membranes. In FIG. 5, figure A was an enlarged view of the part of the boxed part in the FNZ+ figure; figure B was mitochondria labeled with DsRed, and figure C was lysosomes labeled with Lysotraker DeepRed. The results indicate that efflux vesicles wrapped around mitochondria were formed on the surface of cells treated with flunarizine.

### Example 5: Research on Addition Amount of Flunarizine

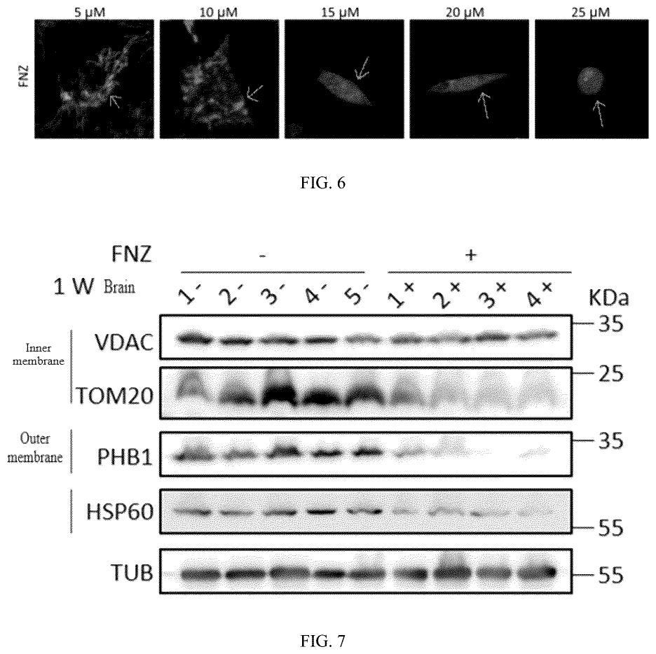

The mito-GFP-labeled neural precursor cells were plated on a 6-well plate, and the surface coverage of the neural precursor cells on the 6-well plate was 80%. After overnight culture, the neural precursor cells were treated with flunarizine for 3 days. The final concentrations of flunarizine were 5, 10, 15, 20, and 25 μM, respectively. Then, the results were detected with a confocal microscope and the efficiency of the mitochondrial clearance was observed. The specific results are illustrated in FIG. 6, in which the parts indicated by the arrows are the neural precursor cells. The mitochondrial morphology was basically not observed in the neural precursor cells treated with flunarizine at the final concentrations of 15 μM, 20 μM, and 25 μM. The results suggest that after 3 days of treatment, flunarizine in the concentration above 10 μM can effectively remove the mitochondria in the cells.

### Example 6: Research on Flunarizine can Effectively Remove Mitochondria in the Brain

Adult ICR mice were injected intraperitoneally with flunarizine (experimental group), and the injection dose of flunarizine was 30 mg/kg, each dose per day, for a total of 7 days. The mice were sacrificed to collect the brain tissues of the mice, and then the brain tissues were homogenized and detected by western blot. The change of the total amount of mitochondria was observed by detecting the content of different types of proteins (such as PHB1 protein and HSP60 protein on the inner membrane, VDAC protein, and TOM20 protein on the outer membrane) on the mitochondria. The specific results are illustrated in FIG. 7, in which the mice that were not injected with flunarizine served as the control group. 5 mice were selected for detection in the control group, and 4 mice were selected for detection in the experimental group. The results suggest that flunarizine can effectively remove the mitochondria in the brains of mice.

In the specification, descriptions referring to the terms “one embodiment”, “some embodiments”, “example”, “specific examples”, or “some examples” mean that specific features, structures, materials or characteristics described in connection with this embodiment or example are included in at least one embodiment or example of the present disclosure. In this specification, the schematic expressions of the above terms are not necessarily directed to the same embodiment or example. Furthermore, the described specific features, structures, materials or characteristics may be combined in any suitable manner in any one or more embodiments or examples. In addition, without conflicting each other, those skilled in the art can integrate and combine different embodiments or examples and features of different embodiments or examples described in this specification.

Although the embodiments of the present disclosure have been shown and described above, it can be understood that the above embodiments are exemplary and should not be construed as limiting the present disclosure, those skilled in the art can make changes, modifications, substitutions and variations to the above embodiments within the scope of the present disclosure.

What is claimed is:

1. A method for removing intracellular mitochondria, the method comprising:
contacting flunarizine with cells to be treated.

2. The method according to claim 1, wherein the mitochondria comprise mitochondria with a DNA mutation and/or mitochondria without a DNA mutation.

3. A method for preventing and/or treating a disease associated with mitochondrial abnormality, the method comprising:
administrating flunarizine to a subject in need thereof.

4. The method according to claim 3, wherein the disease related to mitochondrial abnormality comprises at least one of mitochondrial myopathy, mitochondrial encephalomyopathy, and neurodegenerative disease.

5. The method according to claim 4, wherein the neurodegenerative disease comprises at least one selected from Alzheimer's disease, Parkinson's disease, Huntington's disease, amyotrophic lateral sclerosis, spinocerebellar ataxia, lobar sclerosis, cerebral ischemia, brain injury, and epilepsy.

6. The method according to claim 4, wherein the mitochondrial encephalomyopathy comprises at least one selected from myoclonic epilepsy with red fiber disease, mitochondrial encephalomyopathy with lactic acidosis and stroke-like episodes syndrome, Leber's hereditary optic neuropathy, ophthalmoplegia syndrome, subacute necrotizing encephalopathy, familial primary progressive cerebral gray matter atrophy, kinky-hair syndrome, and retinitis pigmentosa ataxia peripheral neuropathy.

7. The method according to claim 3, wherein a working concentration of the flunarizine ranges from 10 to 30 μM, and preferably, 15 to 25 μM.

8. A single-dose flunarizine formulation, comprising 10 to 30 μM, and preferably, 15 to 25 μM of flunarizine.

9. A method for promoting mitochondrial efflux, comprising:
contacting flunarizine with cells to be treated.

10. A method for controlling a removal amount of mitochondria, comprising:
contacting flunarizine with cells to be treated;
controlling the removal amount of intracellular mitochondria based on a contact time.

11. The method according to claim 10, wherein the contact time is less than 1 day, and the removal amount of the intracellular mitochondria is not higher than ⅓.

12. The method according to claim 10, wherein the reaction time is 1 to 2 days, and the removal amount of the intracellular mitochondria ranges from ⅓ to ½.

13. The method according to claim 10, wherein the reaction time is more than 2 days, and the removal amount of the intracellular mitochondria is higher than ½.

14. The method according to claim 10, wherein the reaction time is more than 3 days, and the intracellular mitochondria are completely removed.

15. The method according to claim 10, wherein a concentration of the flunarizine in a contact environment ranges from 10 to 30 μM, and preferably, 15 to 25 μM.

16. The method according to claim 10, wherein the cells to be treated are plated in a cell culture plate in advance.

17. The method according to claim 10, wherein a surface coverage of the cells to be treated in the cell culture plate ranges from 75% to 85%.

18. The method according to claim 10, wherein the contact time is 0.1 to 5 days, and preferably, 0.5 to 3 days.

19. A cell, with the number of intracellular mitochondria lower than the number of intracellular mitochondria in a normal cell, wherein:
exogenous parkin is not expressed in the cell, and the cell is obtained by the method according to claim 10.
